SEQUENCE LISTING (6 sequences)

SEQ ID NO: 1 - DNA, 27 bp, Homo sapiens
aatggcaacatcaggtcggccatcact

SEQ ID NO: 2 - DNA, 27 bp, Homo sapiens
gctgtgtgtcacagaagtctcgaactc

SEQ ID NO: 3 - DNA, 20 bp, Homo sapiens
tgctggtggaaaaacctcgt

SEQ ID NO: 4 - DNA, 20 bp, Homo sapiens
aaaacccacttcgcctccaa

SEQ ID NO: 5 - DNA, 27 bp, Homo sapiens
tgaaggtcggtgtgaacggattttggc

SEQ ID NO: 6 - DNA, 25 bp, Homo sapiens
tggttcacacccatcacaaacatgg

(19) United States
(12) Patent Application Publication
(10) Pub. No.: US 2023/0131608 A1
(43) Pub. Date: Apr. 27, 2023

(54) COMPOSITION FOR TREATMENT AND PREVENTION OF INFLAMMATORY DISEASES COMPRISING COMPOSITE EXTRACT OF BEET, RED ONION AND PURPLE SWEET POTATO AS ACTIVE INGREDIENTS

(71) Applicant: GUSTAR CO., LTD., Gyeonggi-do (KR)

(72) Inventor: Sujin YANG, Seoul (KR)

(21) Appl. No.: 17/912,592

(22) PCT Filed: Jun. 11, 2020
(86) PCT No.: PCT/KR2020/007593

§ 371 Date: Sep. 19, 2022

(30) Foreign Application Priority Data
Mar. 26, 2020 (KR) 10-2020-0036559

(51) Int. Cl. A61K 36/8962; A61K 36/21; A61K 36/39; A61P 29/00; A23L 33/105

(52) U.S. Cl. CPC A61K 36/8962; A61K 36/21; A61K 36/39; A61P 29/00; A23L 33/105

(57) ABSTRACT

A method for treating or suppressing inflammatory disease includes administering a composition to a subject in need thereof. The composition includes a composite extract of beet, red onion, and purple sweet potato as an active ingredient. The composition has no cytotoxicity, effectively inhibits NO-mediated inflammatory reactions, inhibits the production of IL-6 protein, and inhibits the expression of iNOS COX-2 genes, which are representative inflammation-related genes, thus having an excellent anti-inflammatory effect, and so it can be usefully used for the treatment and prevention of inflammatory diseases in cosmetics, food, and quasi-drug fields. In addition, the composition of the present invention is not only very safe to human body but also excellent in stability.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2020/007593, filed Jun. 11, 2020, which claims priority to the benefit of Korean Patent Application No. 10-2020-0036559 filed in the Korean Intellectual Property Office on Mar. 26, 2020, the entire contents of which are incorporated herein by reference.

### BACKGROUND

### 1. Technical Field

The present invention relates to a composition for the treatment and prevention of inflammatory diseases, the composition containing a composite extract of beet, red onion, and purple sweet potato as active ingredients.

### 2. Background Art

Lipopolysaccharide (LPS), a lipid-polysaccharide complex, is an endotoxin O-antigen found in the cell wall of Gram-negative bacteria, and bacterial LPS typically consists of lipid A (endotoxin), a non-repeating core oligosaccharide, and polysaccharide (O-antigen). The lipid portion (lipid A) forms a composite with the core polysaccharide portion through glycoside bonds. The core portion is linked to a highly immunogenic and variable O-chain polysaccharide, composed of repeating oligosaccharide units, i.e., a third external region, which is the O-antigen region.

On the other hand, nitric oxide (NO) is an important intracellular and intercellular signaling molecule involved in regulating various physiological mechanisms in the immune system. Nitric oxide reacts with superoxide to produce peroxynitrite ions to induce excessive inflammatory activity, and is known to cause such diseases as sepsis, ulcerative colitis, arthritis, and systemic lupus erythematosus (SLE) and Sjogren's syndrome (SS). Reactive oxygen species (ROS) and NO play a very important physiological role as an intermediary in the signaling process.

However, excessive production of endogenous and/or exogenous ROS and NO are related with the onset of inflammatory diseases and cancer. In addition, a redox-sensitive transcription factor, NF-κB, is activated by inflammatory mediators such as oxidative stress, exogenous ROS, and NO. When NF-κB is activated, IκB is phosphorylated by an IκB kinase (IKK) complex, and IκB is ubiquitinated and then degraded. The free NF-κB isolated from IκB migrates to the nucleus, and transcribes genes related to cell growth regulation, apoptosis, immune response, inflammatory response and cancer in the nucleus. In addition, it has been reported that NF-κB plays an important role in the pathogenesis of chronic inflammatory diseases and various human cancers.

Therefore, when a substance that inhibits the generation of NO is searched, the substance can prove to be effective in the prevention and treatment of various inflammatory diseases.

In addition, with regard to various inflammatory regulation factors in cells, when inflammatory cells such as macrophages in the body are activated by inducible nitric oxide synthase (iNOS) or cyclo-oxygenase-2 (COX-2), inflammatory mediators, such as NO, prostaglandin (PG), interleukin-1 beta (IL-1β), tumor necrosis factor-alpha (TNF-α), cytokines and others are produced in large quantities (Jun-Ho Son et. al., Anti-Inflammatory Effect of Ligularia fischeri, Solidago virga-aurea and Aruncus dioicus Complex Extracts in Raw 264.7 Cells, Journal of Life Science, 2011, Vol. 21, No. 5, pp. 678-683). In addition, interleukin-1 (IL-1), which is an inflammatory cytokine, and tumor necrosis factor-α (TNF-α) are induced, and they affect tissues such as muscles, tendons, ligaments, thus causing severe pain. (Fernandes J. C. et al., The Role of Cytokines in Osteoarthritis Pathophysiology, 2002, 39, pp. 237-246).

Non-steroidal anti-inflammatory drugs (NSAIDS), therapeutics that are widely used for the treatment of most inflammatory diseases, exhibit anti-inflammatory action by inhibiting the enzymatic activity of cyclooxygenase (COX) that is involved in the biosynthesis of prostaglandin from arachidonic acid. However, since NSAIDS cause severe side effects such as gastrointestinal disorders, liver disorders, and kidney disorders, besides the main therapeutic action, long-term use of NSAIDS is limited.

Therefore, research has been conducted on the composition for the treatment of inflammatory diseases by using natural plant extracts, such as patent registration No. 10-0532633, patent registration No. 10-1390084, patent registration No. 10-1160488, and patent registration No. 10-1977977. The replacement of NSAIDS is desperately required to develop a new anti-inflammatory analgesic agent exhibiting excellent anti-inflammatory efficacy without any side effects in long-term. That is why research has recently been activated on the development of materials through efficacy verification of natural materials.

Under this background, the inventors of the present invention conducted a study on the combination of natural substances with excellent anti-inflammatory effects, and found out that the composite extract prepared by combining beet extract, red onion extract, and purple sweet potato extract exhibits excellent anti-inflammatory effects, thus completing the present invention.

### SUMMARY

Therefore, the main purpose of the present invention is to provide a pharmaceutical composition for the treatment and prevention of inflammatory diseases, the pharmaceutical composition comprising a composite extract of beet, red onion, and purple sweet potato as active ingredients.

Another purpose of the present invention is to provide a functional food composition for the prevention of inflammatory diseases, the functional food composition comprising a composite extract of beet, red onion, and purple sweet potato as active ingredients.

Another purpose of the present invention is to provide a functional cosmetic composition for the prevention of inflammatory diseases, the functional cosmetic composition comprising a composite extract of beet, red onion, and purple sweet potato as active ingredients.

Other purposes and advantages of the present invention will become more apparent from the following detailed description of the invention, claims and drawings.

According to one aspect of the present invention, the present invention provides a pharmaceutical composition for the treatment and prevention of inflammatory diseases, the pharmaceutical composition comprising a composite extract of beet, red onion, and purple sweet potato as active ingredients.

The pharmaceutical composition for the treatment and prevention of inflammatory diseases, the pharmaceutical composition comprising a composite extract of beet, red onion, and purple sweet potato of the present invention, plays the role of inhibiting inflammation by inhibiting the production of NO, which is involved in the development of inflammation. In addition, the composite extract of beet, red onion, and purple sweet potato of the present invention is capable of inhibiting JNK and ERK, which are phosphorylases, and p38 protein phosphatase through the inhibition of the production of NO in the signaling system mediated by the inflammation-inducing agent LPS, and exhibits an effect of inhibiting the production of IL-6, which is an inflammatory cytokine, and inhibiting the expression of iNOS (inducible NO synthase) and COX-2 genes.

In the present invention, beet (Beta vulgaris) refers to the root of biennial grass of Amaranthaceae family, Centrospermales order, and Dicotyledoneae class, and it is also referred to as geungongchae, hongchaedu and huayeomchae. In addition, in the present invention, red onion means an onion having a purple color. Onions are classified according to the shape of the scaly bulb, the color of the peel, and the taste. The red onion, also referred to as purple onion, containers a more variety of flavonoids, compared with common onions. In addition, in the present invention, the purple sweet potato (jami sweet potato, purple-fleshed sweet potato) means new varieties of sweet potato bred by artificial crossbreeding, such as jami (1998), borami (2000) and shinjami (2001). These new varieties contain a large amount of pigment extracts, and thus not only the epidermal layer but also the whole flesh has a deep purple color. This functional pigment content is 10 to 60 times higher than that of cranberry, and 5 to 7 times higher than that of grape. Therefore, the new varieties have an excellent value as not only a natural source of functional pigment but also a functional food material.

According to one embodiment of the invention, the composition of the present invention may be provided in the form of a pharmaceutical composition for the prevention or treatment of inflammatory diseases.

In the present invention, “treatment” refers to all actions that improve the symptoms of an inflammatory disease or provide benefit by administration of the composition, and “prevention” refers to all actions that inhibit or delay the development of an inflammatory disease by administration of the composition.

As used in the present invention, the term “extract” includes an extract itself and all formulations of extract that can be formed by using the extract, including an extract obtained by the extraction processing of beet, red onion, and purple sweet potato, a diluent or concentrate of the extract, a dried product obtained by drying the extract, a crude purified product or purified product of the extract, or a mixture thereof. The extract of the present invention may be preferably used in the form of a liquid or dry powder after extraction.

In the extraction of the bit, red onion, and purple sweet potato of the present invention, the extraction method is not particularly limited, and the extraction can be performed by any method commonly used in the art. Non-limiting examples of the extraction method include a solvent fractionation method, a centrifugation method, and the like, and these may be performed alone or in combination of two or more methods.

In the present invention, the type of the extraction solvent used to extract the beet, red onion, and purple sweet potato is not particularly limited, and any solvent known in the art may be used. Non-limiting examples of the extraction solvent include water; C1 to C4 lower alcohols such as methanol, ethanol, propyl alcohol, and butyl alcohol; polyhydric alcohols such as glycerin, butylene glycol, and propylene glycol; and hydrocarbon-based solvents such as methyl acetate, ethyl acetate, acetone, benzene, hexane, diethyl ether, and dichloromethane; or a mixture thereof may be used, preferably, water, lower alcohol, 1,3-butylene glycol and ethyl acetate may be used alone or in combination of two or more.

In the present invention, the extract extracted by hot water extraction or cold immersion extraction is filtered to remove floating solid particles, for example, filtered by using nylon and others to filter particles or filtered by using a freeze filtration method, and then the filtered extract may be used as it is or after it is dried by freeze-drying, hot-air drying, or spray drying.

In the pharmaceutical composition for the treatment and prevention of inflammatory diseases of the present invention, the “inflammatory disease” collectively refers to diseases with inflammation as a primary lesion, and in the present invention, the inflammatory disease is an NO-mediated inflammatory disease. Preferably, the NO-mediated inflammatory disease includes atopic dermatitis, psoriasis, sinusitis, rhinitis, conjunctivitis, asthma, dermatitis, inflammatory collagen vascular disease, glomerulonephritis, encephalitis, inflammatory enteritis, chronic obstructive pulmonary disease, sepsis, septic shock, pulmonary fibrosis, undifferentiated spondylarthropathy, undifferentiated arthropathy, arthritis, inflammatory osteolysis, chronic inflammatory diseases caused by viral or bacterial infections, colitis, ulcerative colitis, inflammatory bowel disease, type 1 diabetes, type 2 diabetes, arthritis, rheumatoid arthritis, reactive arthritis, osteoarthritis, scleroderma, osteoporosis, atherosclerosis, myocarditis, endocarditis, pericarditis, cystic fibrosis, Hashimoto's thyroiditis, Graves' disease, leprosy, syphilis, Lyme disease, borreliosis, neurogenic-borreliosis, tuberculosis, sarcoidosis, lupus, chilblain lupus, tuberculous lupus, lupus nephritis, systemic lupus erythematosus, macular degeneration, uveitis, irritable bowel syndrome, Crohn's disease, Sjögren syndrome, fibromyalgia, chronic fatigue syndrome, chronic fatigue immunodeficiency syndrome, myalgic encephalomyelitis, amyotrophic lateral sclerosis, Parkinson's disease, Alzheimer's disease, multiple sclerosis, autism spectrum disorder, attention deficit disorder, attention deficit hyperactivity disorder, and the like.

The composite extract of beet, red onion, and purple sweet potato of the present invention has an excellent effect on the treatment and prevention of inflammatory diseases, specifically, inhibiting the production of NO, which is a direct mediator of inflammatory response, inhibiting the production of IL-6 (interleukin-6) protein, and inhibiting the expression of inducible NO synthase (iNOS) and COX-2 genes.

In the pharmaceutical composition for the treatment and prevention of inflammatory diseases of the present invention, the beet, red onion, and purple sweet potato are preferably mixed at a weight ratio of 1:0.5-1.5:0.5-1.5. In an Example of the present invention, it was confirmed that the composite extract of the beet, red onion, and purple sweet potato has no cytotoxicity, directly inhibits NO production, inhibits IL-6 protein production, and expresses iNOS and COX-2 genes, thus being effective in the treatment and prevention of inflammatory diseases. In particular, it was confirmed that a composite extract prepared by mixing beet, red onion, and purple sweet potato at a ratio of 1:0.5-1.5:0.5-1.5 showed the highest synergistic effect with respect to inflammation inhibition.

The pharmaceutical composition of the present invention comprises a pharmaceutically acceptable carrier. The pharmaceutically acceptable carrier included in the pharmaceutical composition of the present invention is commonly used in formulation, and includes lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate and mineral oil, but is not limited thereto. The pharmaceutical composition of the present invention may further comprise, in addition to the abovementioned components, a lubricant, a wetting agent, a sweetener, a flavoring agent, an emulsifying agent, a suspending agent, a preservative, and the like.

Suitable pharmaceutically acceptable carriers and formulations are described in detail in Remington's Pharmaceutical Sciences (19th ed., 1995).

The pharmaceutical composition of the present invention may be administered orally or parenterally, and in the case of parenteral administration, it may be administered by intravenous injection, subcutaneous injection, intramuscular injection, intraperitoneal injection, transdermal administration, and the like.

Suitable dosages of the pharmaceutical composition of the invention are variously prescribed by factors such as formulation method, mode of administration, patient's age, weight, sex, morbidity, food, time of administration, route of administration, rate of excretion, and response sensitivity. The daily dosage of the pharmaceutical composition of the present invention is, for example, 0.001-100 mg/kg.

The pharmaceutical composition of the present invention is prepared in the form of a unit dose by formulating the same by using a pharmaceutically acceptable carrier and/or excipient according to a method that can be easily implemented by those skilled in the art to which the present invention belongs. Alternatively, it may be manufactured by incorporating the same into a multi-dose container. At that time, the formulation may be in the form of a solution, suspension, syrup or emulsion in an oil or aqueous medium, or may be in the form of extract, powder, granule, tablet or capsule, and may further include a dispersant or stabilizer.

According to another aspect of the present invention, the present invention provides a method for preventing or treating inflammatory diseases, wherein the method comprises administering a pharmaceutical composition for the treatment and prevention of inflammatory diseases to an individual other than humans, the pharmaceutical composition comprising the composite extract of the beet, red onion, and purple sweet potato as an active ingredient. Inflammatory diseases and methods of administration of the present invention are described above. The term “administration” of the present invention means the introduction of a predetermined substance to an individual in an appropriate way. The term “individual” of the present invention refers to all animals, such as rat, mice and livestock, including humans, who may have an inflammatory disease or who may develop an inflammatory disease.

As another aspect, by administering a pharmaceutical composition for the treatment and prevention of inflammatory diseases, the pharmaceutical composition comprising a composite extract of beet, red onion, and purple sweet potato according to the present invention as an active ingredient, an immune response may be suppressed or inflammation may be controlled. Therefore, the present invention provides a method for suppressing inflammatory response of a subject, wherein the method comprises administering a pharmaceutical composition for the treatment and prevention of inflammatory diseases, the pharmaceutical composition comprising a composite extract of the beet, red onion, and purple sweet potato as an active ingredient.

In the present invention, “subject” refers to mammals including cows, dogs, pigs, chickens, sheep, horses, and humans, but is not limited thereto. In addition, preferably, the administration of the composition containing the composite extract of beet, red onion, and purple sweet potato as an active ingredient may be intraperitoneal or intravascular administration, direct administration to a lesion, or administration into a synovial cavity of a joint.

According to another aspect of the present invention, the present invention may be provided in the form of a functional food composition for the prevention of inflammatory diseases, the functional food composition comprising the composite extract of beet, red onion, and purple sweet potato as an active ingredient, and the inflammatory diseases are described above.

The food composition of the present invention comprises, besides the composite extract of beet, red onion, and purple sweet potato, ingredients that are commonly added in the manufacturing of food. For example, food composition of the present invention comprises proteins, carbohydrates, fats, nutrients, seasonings and sweeteners. Examples of the aforementioned carbohydrates include monosaccharides, for example, glucose, fructose, and the like; disaccharides, for example, maltose, sucrose, oligosaccharides, and the like; and polysaccharides, for example, common sugars such as dextrin and cyclodextrin, and sugar alcohols such as xylitol, sorbitol, and erythritol. As a sweetener, natural sweeteners [thaumatin, stevia extract (for example, rebaudioside A, glycyrrhizin, etc.)] and synthetic sweeteners (saccharin, aspartame, etc.) can be used. For example, when the food composition of the present invention is manufactured as a drink agent, the food composition may further comprise, besides the active ingredient of the present invention, citric acid, liquid fructose, sugar, glucose, acetic acid, malic acid, fruit juice, Eucommia ulmoides extract, jujube extract, licorice extract and the like.

In the functional food composition for the treatment and prevention of inflammatory diseases of the present invention, the inflammatory diseases may be NO-mediated inflammatory diseases.

According to another aspect of the present invention, the present invention provides a functional cosmetic composition for the prevention of inflammatory diseases, the functional cosmetic composition comprising the composite extract of beet, red onion, and purple sweet potato as an active ingredient. The composite extract of beet, red onion, and purple sweet potato of the present invention, and the treatment and prevention of inflammatory diseases are described above.

In the functional cosmetic composition for the prevention of inflammatory diseases of the present invention, the composition may be manufactured as a formulation selected from the group consisting of solution, external ointment, cream, foam, nutrient tonic, softening tonic, mask pack, softening water, milky lotion, makeup base, essence, soap, liquid detergent, bathing agent, sunscreen cream, sun oil, suspension, emulsion, paste, gel, lotion, powder, soap, surfactant-containing cleaning, oil, powder foundation, emulsion foundation, wax foundation, patch and spray, but is not limited thereto.

In addition, the cosmetic composition of the present invention may further comprise one or more cosmetically acceptable carriers to be blended with general skin cosmetics, and common components, for example, oil, water, surfactant, moisturizing agent, lower alcohol, thickener, chelating agent, pigment, preservative, fragrances and the like may be appropriately blended, but are not limited thereto.

The cosmetically acceptable carrier included in the cosmetic composition of the present invention varies depending on the formulation.

When the formulation of the present invention is an ointment, paste, cream or gel, as a carrier component, animal oil, vegetable oil, wax, paraffin, starch, tragacanth, cellulose derivative, polyethylene glycol, silicone, bentonite, silica, talc, zinc oxide or a mixture thereof can be used.

When the formulation of the present invention is a powder or spray, as a carrier component, lactose, talc, silica, aluminum hydroxide, calcium silicate, polyamide powder or a mixture thereof can be used as a carrier component, especially in the case of a spray, a propellent such as chlorofluorohydrocarbon, propane/butane or dimethyl ether may be further included.

When the formulation of the present invention is a solution or emulsion, as a carrier component, a solvent, solubilizing agent or emulsifying agent is used, for example, water, ethanol, isopropanol, ethyl carbonate, ethyl acetate, benzyl alcohol, benzyl benzoate, propylene glycol, 1,3-butyl glycol oil can be used, and in particular, cottonseed oil, peanut oil, corn seed oil, olive oil, castor oil and sesame oil, glycerol aliphatic esters, polyethylene glycol or fatty acids of sorbitan can be used.

When the formulation of the present invention is a suspension, as a carrier component, liquid diluents such as water, ethanol and propylene glycol; suspending agents such as ethoxylated isostearyl alcohol, polyoxyethylene sorbitol ester and polyoxyethylene sorbitan ester; microcrystalline cellulose; aluminum metahydroxide; bentonite; agar or tragacanth and the like can be used.

When the formulation of the present invention is a soap, as a carrier component, alkali metal salt of fatty acid, fatty acid hemiester salt, fatty acid protein hydrolysate, isethionate, lanolin derivative, aliphatic alcohol, vegetable oil, glycerol, sugar and the like can be used.

In the cosmetic composition of the present invention, the composite extract of beet, red onion, and purple sweet potato may be included specifically in dry weight in an amount of 0.0001% to 50% by weight of the total weight of the cosmetic composition, and more specifically, 0.0005 to 10% by weight. Within the above range, there is an advantage of exhibiting excellent efficacy on the treatment and prevention of inflammatory diseases, and there is an advantage that the formulation of the composition is stabilized.

The features and advantages of the present invention are summarized as follows:

(1) The present invention provides a composition for the treatment and prevention of inflammatory diseases, the composition comprising a composite extract of beet, red onion, and purple sweet potato as an active ingredient.

(2) The composition for the treatment and prevention of inflammatory diseases of the present invention has no cytotoxicity, effectively inhibits NO-mediated inflammatory reactions, inhibits the production of IL-6 protein, and inhibits the expression of iNOS COX-2 genes, which are representative inflammation-related genes, thus having an excellent anti-inflammatory effect, and so it can be usefully used for the treatment and prevention of inflammatory diseases in cosmetics, food, and quasi-drug fields.

(3) In addition, the composition of the present invention is not only very safe to human body but also excellent in stability.

### DETAILED DESCRIPTION OF THE DRAWINGS

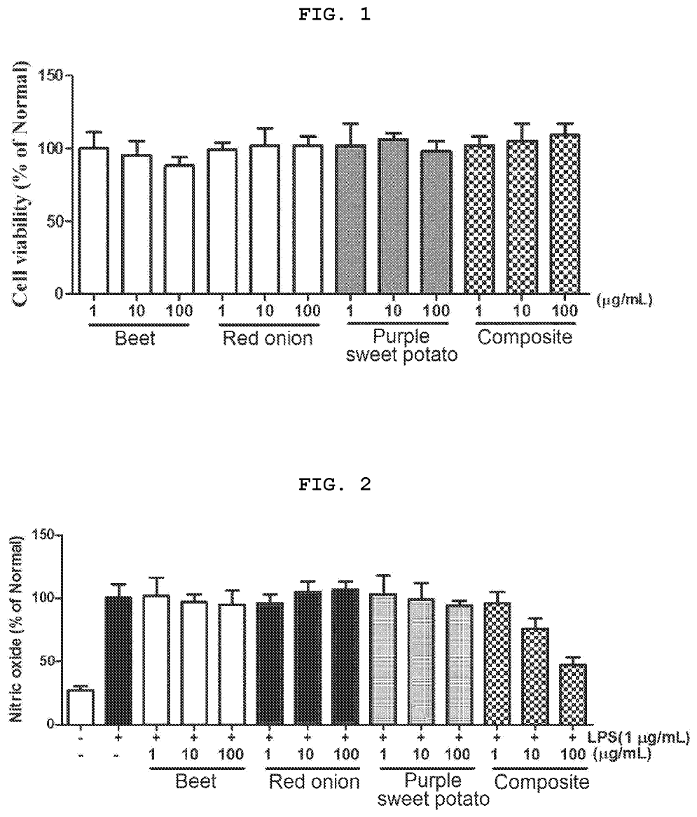

FIG. 1 is a graph showing the analysis of cell proliferation of human keratinocytes (HaCaT) according to the composite extract according to the treatment of beet, red onion, and purple sweet potato of the present invention.

FIG. 2 is a graph showing the measurement of the change of the NO production in the mouse macrophage cell line (RAW264.7) according to the treatment of the composite extract of beet, red onion, and purple sweet potato of the present invention.

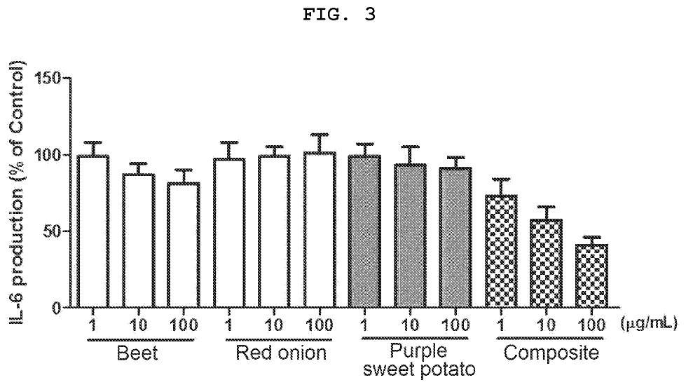

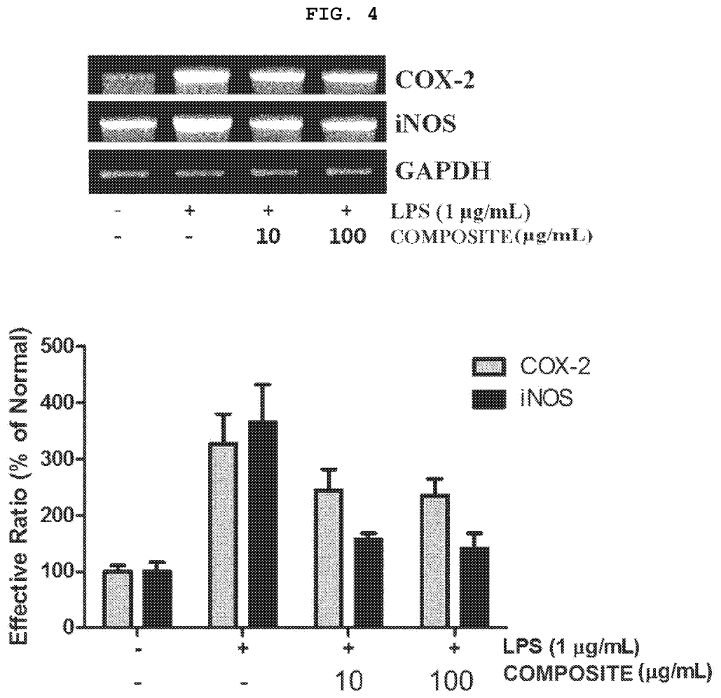

FIG. 3 is a graph showing the measurement of the change of the IL-6 protein production in human keratinocytes (HaCaT) according to the treatment of the composite extract of beet, red onion, and purple sweet potato of the present invention.

FIG. 4 is a diagram showing the results of measuring the change of the expression of iNOS and COX-2 genes according to the treatment of the composite extract of beet, red onion, and purple sweet potato of the present invention.

### DETAILED DESCRIPTION

Hereinafter, the present invention will be described in more detail through Examples. Since these Examples are only for illustrating the present invention, the scope of the present invention is not to be construed as being limited by these examples.

### Example 1. Preparation of Extract of Beet, Red Onion, and Purple Sweet Potato

After pulverizing 100 g of dried beet, 3 L of 50% ethanol was used as a solvent and the resulting mixture was subject to heated reflux extraction for 12 hours and cold immersion, and then filtered through a filter paper having a 1.2 μm permeation size to obtain a filtrate. The obtained filtrate was concentrated under reduced pressure and dried under reduced pressure to prepare dried beet powder (Preparation Example 1).

Dried powder of red onion (Preparation Example 2) and dried powder of purple sweet potato (Preparation Example 3) were prepared by the same method as the preparation of the beet extract.

In addition, the powder of the beet, red onion, and purple sweet potato was mixed at a weight ratio of 1:1:1 to prepare a composite extract thereof (Preparation Example 4).

### Example 2. Cytotoxicity Experiment

The effect of the beet, red onion, and purple sweet potato extract obtained in Example 1 on the growth of human keratinocytes (HaCaT) was confirmed by the method described below.

First, human keratinocytes were inoculated at a concentration of 1×10⁵ in a 24-well cell culture dish in Dulbeccos modified Eagles medium (DMEM), which is an exclusive medium containing 10% fetal bovine serum (FBS, Cambrex), and then incubated at 37° C. under wet conditions with 5% CO₂ for 24 hours. Thereafter, the medium was removed, and then the cells were treated with the single extracts of beet, red onion, and purple sweet potato of Example 1 diluted with serum-free DMEM medium and the composite extract thereof (weight ratio 1:1:1) at different concentrations (1, 10, and 100 μg/mL) and incubated for 24 hours. After 24 hours, the cells were treated with 1 mg/mL of MTT was treated and after 2 hours, formazan produced by the MTT treatment with the cells was dissolved in DMSO to measure light absorbance at 570 nm.

As a result, it was confirmed that the single extracts of beet, red onion, and purple sweet potato of Example 1 and the composite extract thereof did not significantly affect the cell viability of human keratinocytes; rather it was shown that the composite extraction of beet, red onion, and purple sweet potato promoted the survival of human keratinocytes at concentrations of 1, 10, and 100 μg/mL. The results described above confirmed that the single and composite extracts of beet, red onion, and purple sweet potato did not show toxicity to human body (see FIG. 1).

### Example 3. Inhibition of NO Production

The change of NO production was measured to measure the anti-inflammatory effect of the single extracts of beet, red onion, and purple sweet potato and the composite extracts thereof. NO is a representative cytotoxic substance involved in causing inflammation.

After preparing RAW264.7 cells, which are mouse macrophages, LPS (1 μg/mL) was treated as a cell activity and inflammatory factor in all groups except the normal group, and after culturing for 24 hours, the cell culture solution was collected.

The NO donor and the Griess reagent were treated together, and the NO appearing over time was measured to quantify the NO inhibitory capacity.

In the experiment, to quantify the amount of NO observed at a specified time, the cells were treated with the single extracts of beet, red onion, and purple sweet potato and the composite extract thereof (mixed at a weight ratio of 1:1:1) at concentrations of 1, 10, and 100 ug/mL and incubated at 37° C. for 2 hours, and then the Griess reagent, a reagent for detecting NO, was mixed to measure the light absorbance at 540 nm

As a result, it was found that the production of NO induced by LPS was inhibited in a concentration-dependent manner in the groups to which the composite extract comprising the 3 ingredients of beet, red onion, and purple sweet potato was added, and the production of NO was inhibited up to 53% at concentration of 100 ug/mL, compared with the untreated group. On the contrary, in the groups treated with single extracts of beet, red onion, and purple sweet potato, the NO production inhibitory activity was negligible. Compared with the groups treated with the single extracts of beet, red onion, and purple sweet potato, the group treated with the 3-ingredient composite extract thereof showed 51%, 57%, and 50% improved NO inhibitory activity, respectively.

Through the experimental results described above, it was confirmed that the 3-ingredient composite extract of beet, red onion, and purple sweet potato of the present invention has a synergic effect of concentration-dependently inhibiting the production of NO, which mediates an inflammatory reaction (FIG. 2).

### Example 4. Inhibition of IL-6 Protein Expression

In the inflammatory response mechanism caused by cell damage, inflammatory mediators such as IL-6 are involved, the inflammatory response is amplified due to the action of the inflammatory mediators, and when the expression of IL-6 is inhibited, the inflammatory response can be effectively blocked.

To verify the effect of the composite extract of beet, red onion, and purple sweet potato on the expression of IL-6 protein, an experiment was performed on human keratinocytes (HaCaT, Human immortalized keratinocyte). The HaCaT cells were inoculated in a 96-well plate at a concentration of 1.0×10⁵, and incubated at 37° C. under wet conditions with 5% CO₂ for 24 hours. Thereafter, after washing the cells with PBS solution, 1 μg/ml of LPS was treated in each well either alone or with samples of various concentrations, and then incubated for 24 hours. In the normal control group, only the medium was treated.

After the incubation is finished, the supernatant was taken and the amount of the produced IL-6 protein was analyzed by using an enzyme-linked immunosorbent assay (ELISA) kit.

As a result, it was found that the production of IL-6 protein induced by LPS was inhibited in a concentration-dependent manner in the groups to which the 3-ingredient composite extract of beet, red onion, and purple sweet potato was added, and the production of IL-6 protein was inhibited up to 58% at concentration of 100 ug/mL, compared with the untreated group. On the contrary, in the groups treated with single extracts of beet, red onion, and purple sweet potato, the IL-6 protein production inhibitory activity was negligible. Compared with the groups treated with the single extracts of beet, red onion, and purple sweet potato, the group treated with the 3-ingredient composite extract showed 50%, 60%, and 55% improved NO IL-6 protein production inhibitory activity, respectively.

Through the results described above, it was confirmed that the composite extract of beet, red onion, and purple sweet potato exhibited a significantly improved IL-6 protein production inhibitory effect, compared to the groups treated with the single extracts thereof, and had a synergic effect was due to the combination of beet, red onion, and purple sweet potato.

### Example 5. Effect of Controlling Inflammation-Inducing Factors

With the composite extract of beet, red onion, and purple sweet potato, which was found to have the best inhibitory effect on the production of NO and the production of IL-6 protein in Examples 3 and 4, the effect of regulating gene expression was verified with respect to iNOS and COX-2 genes, which are inflammation-causing factors.

First, human keratinocytes (HaCaT) were inoculated at a concentration of 1×10⁵ in a 24-well cell culture dish in Dulbeccos modified Eagles medium (DMEM), which is an exclusive medium containing 10% fetal bovine serum (FBS, Cambrex), and then incubated at 37° C. under wet conditions with 5% CO₂ for 24 hours. Thereafter, the medium was removed, and then the cells were treated with samples prepared by diluting the composite extract of beet, red onion, and purple sweet potato (weight ratio 1:1:1) of Example 1 with serum-free DMEM medium at concentrations of 10 and 100 μg/mL and at the same time, with a new medium containing LPS (μg/mL), which is known as an endotoxin.

The experiment was performed by the RT-PCR method to confirm the mRNA expression levels of iNOS and COX-2. The used primers are described below:
iNOS: Forward 5′-AAT GGC AAC ATC AGG TCG GCC ATC ACT-3′(SEQ ID NO:1), Reverse 5′-GCT GTG TGT CAC AGA AGT CTC GAA CTC-3′(SEQ ID NO:2)
COX-2: Forward 5′-TGC TGG TGG AAA AAC CTC GT-3′ (SEQ ID NO:3), Reverse 5′-AAA ACC CAC TTC GCC TCC AA-3′(SEQ ID NO:4)
GAPDH: Forward 5′-TGA AGG TCG GTG TGA ACG GAT TTT GGC-3′ (SEQ ID NO:5), Reverse 5′-TGG TTC ACA CCC ATC ACA AAC ATG G-3′(SEQ ID NO:6).

RT-PCR was performed by using the primers described above, and the results are summarized in FIG. 4.

As a result, with regard to the effect of the composite extract of beet, red onion, and purple sweet potato for inhibiting iNOS and COX-2 expression, compared with the negative control group, the iNOS expression inhibitory effect was improved by 57% and 61% at the concentrations of 10 and 100 μg/mL, respectively, and the COX-2 expression inhibitory effect was improved by 25% and 28%, respectively (see FIG. 4).

Through the results described above, it was confirmed that the composition for the treatment and prevention of inflammatory diseases, the composition comprising the composite extract of beet, red onion, and purple sweet potato of the present invention, has no cytotoxicity, and directly inhibits the production of NO and IL-6 protein, and inhibits the expression of iNOS and IL-6 genes, thus having an effect of treating and preventing inflammatory diseases.

Since the specific parts of the present invention have been described above in detail, it is clear that for those skilled in the art, this specific description is only a preferred embodiment, and the scope of the present invention is not limited thereto.

Therefore, the substantial scope of the present invention will be defined by the appended claims and their equivalents.

A sequence listing electronically submitted with the present application on Sep. 19, 2022 as an ASCII text file named 20220919_Q90522YG03_TU_SEQ.TXT, created on Sep. 19, 2022 and having a size of 1,206 bytes, is incorporated herein by reference in its entirety.

1: A method for treating or suppressing inflammatory disease, the method comprising administering to a subject in need thereof a composition comprising a composite extract of beet, red onion, and purple sweet potato as an active ingredient.

2: The method of claim 1, wherein the inflammatory disease is nitrogen monoxide (NO)-mediated inflammatory disease.

3: A method for inhibiting production of IL-6 (interleukin-6) protein and expression of inducible NO synthase (iNOS) and COX-2 genes in a subject, the method comprising administering to a subject in need thereof.

4: The method of claim 1, wherein the beet, red onion, and purple sweet potato are mixed at a weight ratio of 1:0.5-1.5:0.5-1.5.

5. (canceled)

6: The method of claim 1, wherein the composition is a functional food composition.

7. (canceled)

8: The method of claim 1, wherein the composition is a pharmaceutical composition.
